(19) United States
(12) Patent Application Publication
(10) Pub. No.: US 2015/0140624 A1
(43) Pub. Date: May 21, 2015

(54) PRODUCTION OF LACTIC ACID FROM FERMENTATIONS USING MIXED BACTERIAL CULTURES

(71) Applicant: Fengjie Cui, Zhenjiang (CN)

(72) Inventor: Fengjie Cui, Zhenjiang (CN)

(73) Assignee: Jiangsu University, Zhenjiang, Jiangsu (CN)

(21) Appl. No.: 14/408,010

(22) PCT Filed: Jun. 15, 2012
(86) PCT No.: PCT/CN2012/076993

§ 371 Date: Dec. 15, 2014

(51) Int. Cl. C12P 7/56; C12M 1/00

(52) U.S. Cl. CPC C12P 7/56; C12M 47/12; C12M 45/09; C12M 47/10; C12P 2203/00
USPC 435/139; 435/297.2

(57) ABSTRACT

A method and system for producing lactic acid from biomass materials uses a mixed bacteria culture of at least one homofermentative lactic acid bacteria and at least one heterofermentative lactic acid bacteria in an integrated production system to increase the productivity and yield of lactic acid.

### BACKGROUND

Organic acids are organic compounds with acidic properties. The most common organic acids are the carboxylic acids, whose acidity is associated with their carboxyl group —COOH. Some examples of common organic acids include lactic acid, acetic acid, formic acid, citric acid and alcohols. The organic acid, lactic acid (2-hydroxypropanoic acid, CH₃CHOHCOOH), is the most widely occurring carboxylic acid in nature and plays a role in many biochemical processes. Lactic acid has many applications in the food, chemical, textile, and pharmaceutical industries, and recently there has been an increasing demand for lactic acid for the manufacture of the biodegradable polymer, polylactic acid (PLA). Because of concerns over petroleum-based plastics, such as the environmental impact, health issues, and the rising price and supply of petroleum, the use and development of bioplastics synthesized from corn, soy, sugar cane, and other crops is on the rise. Most of the bioplastic that is now being produced is polymerized lactic acid (PLA). PLA production releases fewer toxic substances than making petroleum plastic and uses less energy, and PLA plastic can be composted, incinerated or recycled.

The current worldwide demand for lactic acid is estimated to be about 130,000 to about 150,000 metric tons per year. Starchy materials are the main substrate for bioproduction of lactic acid and other organic acids. Organic acids may be produced from corn and other starchy plants using microbial fermentation technology. Organic acid feedstocks may first be converted into fermentable sugars, and then further converted into biochemical or diesel products. The fermentable sugars are generally a mixture of six-carbon (C6) sugars, such as glucose, and five-carbon (C5) sugars, such as xylose and arabinose.

Most of the microorganisms used in organic acid fermentation can only utilize the glucose derived from the cellulose fraction, while xylose and other hemicellulose derived sugars cannot be used directly. For example, the homofermentative strain Lactobacillus rhamnosus ferments only the hexoses (glucose, etc.) but no pentoses (xylose, arabinose, etc.). On the other hand, the heterofermentative Lactobacillus Pentosus converts hexoses and pentoses simultaneously into lactic acid, acetic acid and ethanol. Lactic acid and other substances (acetic acid and/or ethanol) produced by heterofermentative lactic acid bacteria are the main end products, and the compositions and ratios vary with the microorganisms and fermentation conditions.

There remains a need for efficiently converting both C5 and C6 sugars of biomass feedstocks into bioproducts with minimal by-products, thereby providing for a more profitable and economical production of bioproducts such as organic acids.

### SUMMARY

Presently disclosed is a system and method for producing organic acids from biomass material with a mixed culture of bacteria. In an embodiment, the system may be an integrated system, and in an additional embodiment the biomass material may be non-food biomass material.

In an embodiment, a method for producing lactic acid from biomass material includes hydrolyzing the biomass material to form a mixture of monosaccharides, and fermenting the monosaccharides in a fermenter with a fermentation broth of a mixed bacteria culture to produce lactic acid and acetic acid. The mixed bacteria culture includes at least one homofermentative lactic acid bacteria comprising Lactobacillus rhamnosus, Lactobacillus delbrueckii, Lactobacillus casei, Lactobacillus acideophilus, Lactobacillus bulgaricus, or combinations thereof, and at least one heterofermentative lactic acid bacteria comprising Lactobacillus pentosus, Lactobacillus brevis, Lactobacillus lactis or combinations thereof. The method also includes converting at least about 90% of the monosaccharides to lactic acid and acetic acid, recovering lactic acid from the fermentation broth at a yield of at least about 0.65 gram lactic acid per gram of monosaccharides, and recovering acetic acid at a yield of at most about 0.065 gram acetic acid per gram of monosaccharides.

In an addition embodiment, a method for producing lactic acid from non-food biomass material includes hydrolyzing non-food biomass material to form a mixture of monosaccharides and fermenting the monosaccharides in a fermenter with a fermentation broth of a mixed bacteria culture to produce lactic acid and co-produced acetic acid. The mixed bacteria culture includes at least one homofermentative lactic acid bacteria and at least one heterofermentative bacteria, wherein the at least one homofermentative lactic acid bacteria comprises Lactobacillus rhamnosus, and the at least one heterofermentative lactic acid bacteria comprises Lactobacillus pentosus. The method also includes recovering acetic acid from the fermentation broth.

In an embodiment, a system for producing lactic acid from biomass material includes a first supply reservoir configured for providing hydrolyzed biomass material, a second supply reservoir configured for providing a mixed bacteria culture medium of at least one homofermentative lactic acid bacteria and at least one heterofermentative lactic acid bacteria, a fermenter configured for receiving the hydrolyzed biomass material and the mixed bacteria culture medium for fermentation of the hydrolyzed biomass material with the mixed bacteria in a resultant fermentation broth, a filter configured for receiving the fermentation broth from the fermenter and separating from the fermentation broth any lactic acid and acetic acid produced by the at least one homofermentative lactic acid bacteria and the at least one heterofermentative lactic acid bacteria, a collector configured for receiving the lactic acid and acetic acid from the filter, and at least one pump for feeding hydrolyzed biomass material from the supply reservoir to the fermenter, feeding the mixed bacteria culture to the fermenter, feeding fermentation broth from the fermenter to the filter, returning at least a portion of the fermentation broth from the filter back to the fermenter, or combinations thereof.

In an embodiment, a kit for producing lactic acid from hydrolyzed biomass materials containing C₅ and C₆ sugars includes at least one homofermentative lactic acid bacteria and at least one heterofermentative lactic acid bacteria for being combined together in a fermentation broth to convert a substantial portion of the C₅ and C₆ sugars into lactic acid, wherein the at least one homofermentative lactic acid bacteria comprises Lactobacillus rhamnosus, and the at least one heterofermentative lactic acid bacteria comprises Lactobacillus pentosus.

### BRIEF DESCRIPTION OF THE FIGURES

FIG. 1 depicts the enzymatic breakdown of cellulose into glucose monomers according to an embodiment.

FIG. 2 depicts the enzymatic breakdown of a hemicellulose into xylose monomers according to an embodiment.

FIG. 3 is an illustrative flow-chart representing one method for producing lactic acid according to an embodiment.

FIG. 4 is an illustrative flow-chart representing an alternative method for producing lactic acid according to an embodiment.

FIG. 5 is an illustrative flow-chart representing another method for producing lactic acid according to an embodiment.

FIG. 6 is a representative system for the production of organic acids from biomass according to an embodiment.

FIGS. 7A-7D show a comparison of results using different bacteria cultures and conditions according to an embodiment.

FIG. 8 shows production and consumption results of a semi-continuous fermentation using the Integrated Production System according to an embodiment.

### DETAILED DESCRIPTION

Lactic acid, of a variety of purity levels, is used in food and beverages, cosmetics, pharmaceutical, biodegradable plastics and other chemical sectors. Over the last few years, demand for lactic acid in industrial applications has far surpassed the demand in food and beverages market. The industrial application market is set to become one of the largest consumers of lactic acid and is expected to consume over half of the world lactic acid production in the near future. Lactic acid based biodegradable polymers followed by lactate solvents are likely to drive the demand for lactic acid.

Biodegradable plastics represent the fastest growing end-use application for lactic acid. With demand for lactic acid-based biopolymers, such as poly lactic acid polymers, expanding at the cost of conventional polymers on counts of environmental friendliness, easy recyclability and cost-effectiveness, emergence of new lucrative opportunities are portended for lactic acid consumption in the coming years.

Biomass materials provide one source for the production of organic acids, such as lactic acid. Biomass materials may be broken down into simple sugars, which may be converted into organic acids by bacterial fermentation. Biomass materials are carbon, hydrogen and oxygen based, and encompass a wide variety of materials including plants, wood, garbage, paper, crops and waste products from processes which use any of these materials. Some waste stream materials include forest residues, municipal solid wastes, waste paper and crop residues. So as not to compete for food sources, organic acids may be produced from the waste stream materials. Some examples of non-food biomass materials may include, but are not limited to, sawdust, corn stover, wheat straw, rice straw, switchgrass, bagasse, poplar wood, paper mill waste and municipal paper waste.

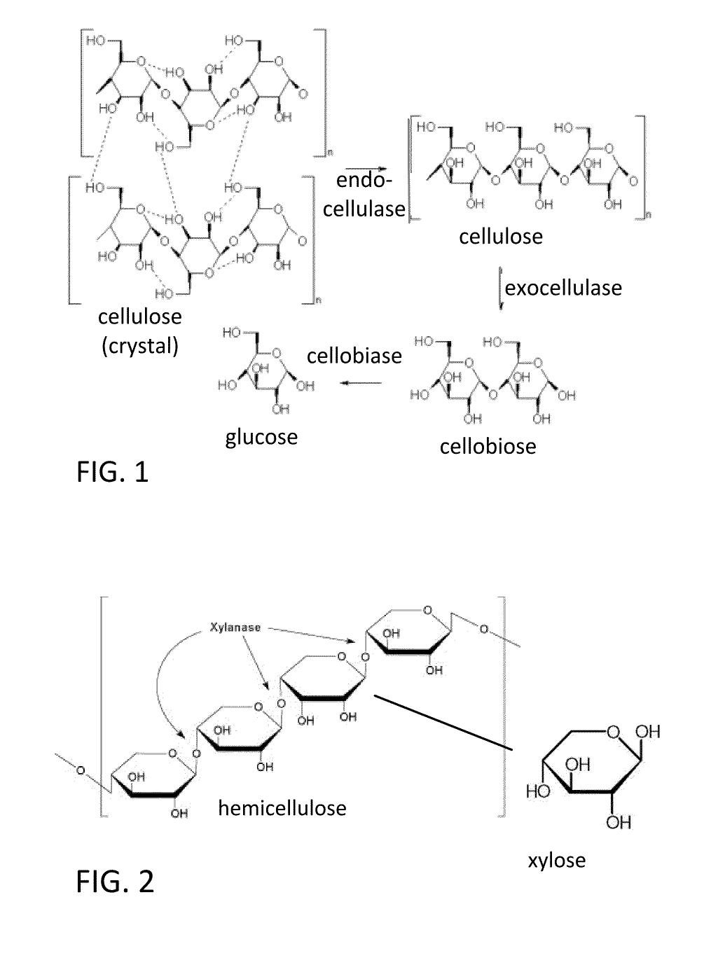

The main components of biomass materials are cellulose (FIG. 1), which may be about 36-42% of dry weight of non-food biomass feedstock, and hemicellulose (FIG. 2), which may be about 21-25% of dry weight of non-food biomass feedstock. As depicted in FIGS. 1 and 2, cellulose and hemicellulose are both formed from sugar monomers. Cellulose is formed from the 6-carbon (C6) sugar glucose, and hemicellulose is formed from both C6 sugars and 5-carbon (C5) sugars. Hemicellulose monomers may include glucuronic acid, galactose, mannose, rhamnose, arabinose, most of the D-pentose sugars and small amounts of L-sugars, with xylose being present in the largest amount.

It has been estimated that more efficient utilization of both C5/C6 sugars from cellulose and hemicellulose components may possibly give a cost saving of up to about 25% for production of organic acids or fuels such as lactic acid and bioethanol.

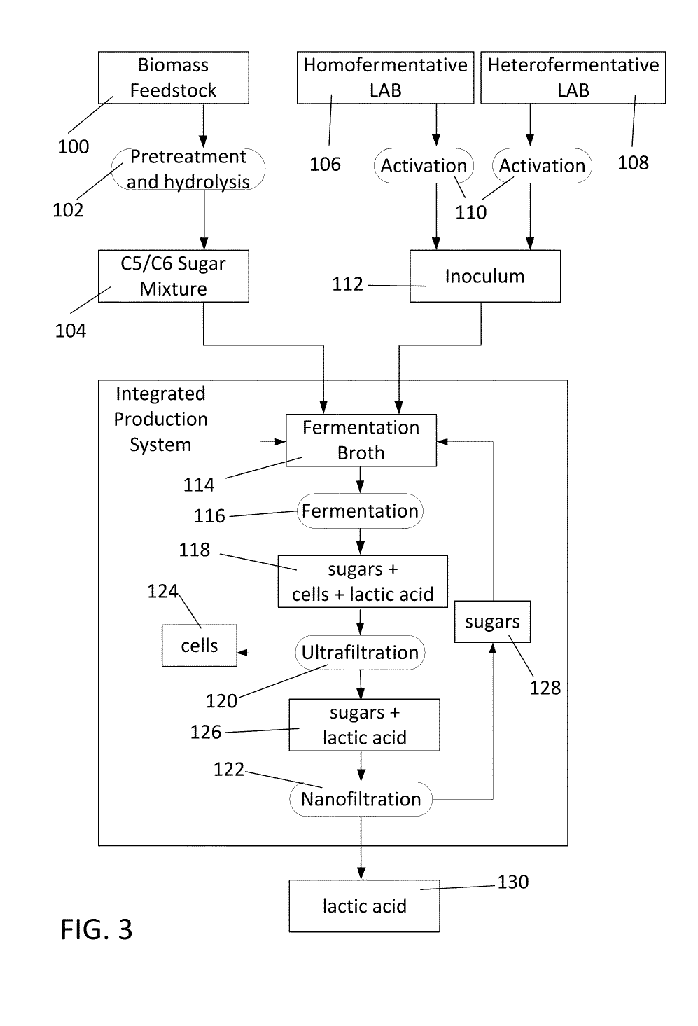

In an embodiment, with reference to FIGS. 3-5, biomass materials as a biomass feedstock 100 may be subjected to a pre-treatment process 102 to remove an additional lignin component. The pre-treating step may include placing the biomass material 100 into contact with an acid or a base in a reaction vessel to break down any lignins in the biomass material. In one pre-treatment process, the biomass may be contacted with an acid at a temperature from about 60° C. to about 170° C. In an embodiment, the biomass material may be contacted with about 0.5 M to about 1.5 M of at least one of sulfuric acid, hydrochloric acid and nitric acid at a temperature from about 80° C. to about 150° C. for about 2 to about 80 minutes. In an alternative pre-treatment process, the biomass may be contacted with a base at temperature from about 25° C. to about 80° C. In an embodiment, the biomass material may be contacted with about 0.1M to about 2.0 M of at least one of sodium hydroxide, potassium hydroxide or calcium hydroxide at temperature from about 25° C. to about 60° C. for about 0.5 to about 5 hours.

For production of organic acids, biomass materials may be broken down by hydrolysis 102 into simple sugar monomers, which may then be converted into organic acids. The biomass material 100 may be placed in a reaction vessel with at least one enzyme that is able to break down the larger biomass components, cellulose and hemicellulose, into the sugar monomers. FIG. 1 depicts one embodiment for the hydrolysis of cellulose into glucose, while FIG. 2 depicts one embodiment for the hydrolysis of hemicellulose into xylose.

Cellulase enzymes encompass a broad group of enzymes which may provide for hydrolysis of cellulose in several different ways. As shown in FIG. 1, cellulose may be contacted with an endocellulose to break bonds between individual strands of cellulose, an exocellulase to break down cellulose into cellobiose, and a cellobiase to break down the cellobiose into individual glucose monomers. Some examples of the groups of cellulase enzymes which may be used in an embodiment include endocellulase (EC 3.2.1.4), exocellulase (EC 3.2.1.91) and cellobiase (EC 3.2.1.21).

FIG. 2 depicts a hemicellulose of xylose and the points at which a xylanase enzyme may hydrolyze linkages to form the xylose monomer. Xylanase enzymes encompass a class of enzymes which may provide for hydrolysis of hemicellulose bonds. Some examples of xylanases which may be used in an embodiment include endo-1,4-β-xylanase (EC 3.2.1.8), exo-1,3-β-xylosidase (EC 3.2.1.72), and exo-1,4-β-xylosidase (EC 3.2.1.37).

In an embodiment, specialized enzyme mixtures, such as Spezyme® CP (cellulase activity of 50 FPU/mL from Genencor International, Palo Alto, Calif., USA) may be used for the hydrolysis step. Spezyme® CP may effectively hydrolyze both cellulose and hemicelluloses. Other methods of breaking down or hydrolyzing cellulose and hemicelluloses into their constituent C5 and C6 sugar monomers 104 may also be used.

The C5/C6 sugar monomers 104, which may include mostly glucose and xylose, may be converted into organic acids or biofuels by the use of appropriate bacteria. In an embodiment for the production of lactic acid, the bacteria may be homofermentative lactic acid bacteria (LAB) 106 and heterofermentative LAB 108. Glucose may be converted to lactic acid by homofermentative LAB via the Embden-Meyerhof Pathway (EMP). Some examples of homofermentative LAB may include, but are not limited to Lactobacillus rhamnosus, L. delbrueckii, L. casei, L. acideophilus or L. bulgaricus. Xylose and a limited amount of glucose may be converted to lactic acid and acetic acid by heterofermentative LAB via the phosphoketolase (PK) pathway. Some examples of heterofermentative LAB may include Lactobacillus Pentosus, L. brevis or L. lactis.

By using a mixture of at least one homofermentative LAB 106 and at least one heterofermentative LAB 108 for the fermentation of the sugar mix 104 a higher efficiency and productivity of lactic acid may be obtained. More of the C5/C6 sugars may be converted into the desired target product, lactic acid, with a reduced conversion of the sugars into the by-product, acetic acid. With a mixed culture, and processes as discussed below, at least about 90% of the monosaccharides in the C5/C6 sugar mixture may be converted. In addition, a yield of at least about 0.65 g and as high as about 0.92 g lactic acid per g of monosaccharides may be attained, while simultaneously also only producing a yield of at most about 0.065 g acetic acid per g of monosaccharides. Further, the productivity of lactic acid may be about 0.50 gram per Liter-hour (g/L·h) to about 0.89 g/L·h. with a batch production method, and may be about 2.5 g/L·h. to about 3.1 g/L·h. with a continuous production method as described further below.

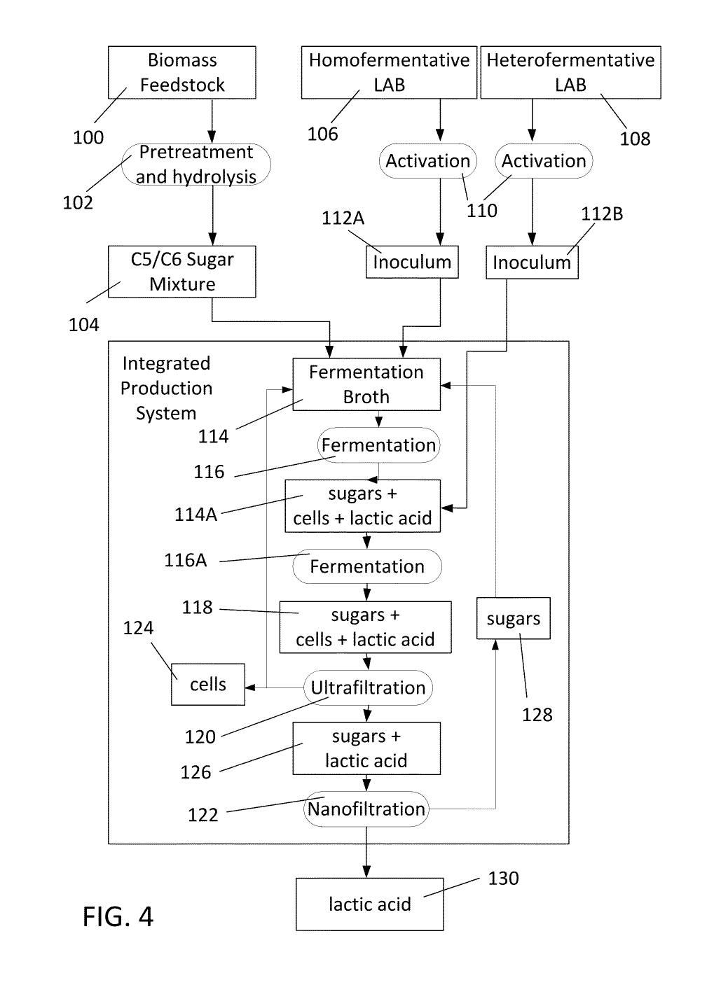

In preparation for inoculation of the fermentation broth, the bacteria 106, 108 may be activated 110 by growth in a nutrient broth or media. One type of nutrient source which may be used for activating the cultures is Man Regosa Sharpe (MRS) media. In an embodiment, the activated bacterial cultures may be combined into a single inoculum 112 as shown in FIG. 3, or alternatively, as depicted in FIG. 4, the bacteria cultures may be kept as separate inoculums 112A and 112B. The sugar mixture 104 and a bacteria inoculum 112 (or inoculums 112A and 112B) may be combined in a fermentation broth 114 to ferment the sugars and produce lactic acid. In one embodiment, and as depicted in FIG. 3 the bacteria may be added together for simultaneous cultivation of the monosaccharides into organic acids. Or alternatively, as depicted in FIG. 4, a two-stage cultivation strategy may be used wherein at least one bacteria strain may be added prior to others to convert a first portion of the monosaccharides into organic acids, and then, after a period of time, at least one additional bacteria strain may be added to convert additional monosaccharides into organic acids. In an embodiment as shown in FIG. 4, the homofermentative LAB may be added first to convert at least a portion of the glucose into lactic acid, and after a period of time, which may be for example, about 12 hours, the heterofermentative bacteria may be added to convert an additional portion of the monosaccharides into lactic acid.

In an embodiment, the sugars may be fermented with the bacteria in a batch system, wherein the sugars and bacteria are combined in a fermenter and allowed to ferment for a period of time. In an embodiment, such a batch process may include at least one additional replacement of the broth/media in the fermenter to provide an increase in yield. The broth containing the organic acids may be separated from the bacteria and any undigested sugars, and the organic acids may be separated from the broth.

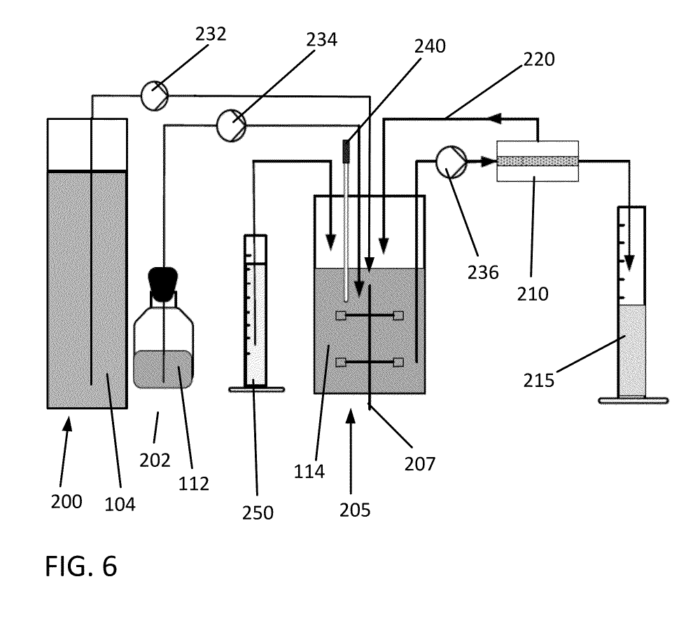

As an alternative to the batch system, an embodiment which may provide an increased productivity for large scale production may be an integrated production system (IPS) allowing for an essentially continuous flow-through production. An IPS may include supply sources, a fermenter, a separation device, and a collection container. One embodiment of such an IPS may be represented by the illustration in FIG. 6. In an embodiment, an IPS for producing organic acids from biomass materials may include a first supply reservoir 200 for the hydrolyzed biomass material, which material, in correspondence with the diagram of FIG. 3, may be the C5/C6 sugar mixture 104. A second supply reservoir 202 may be provided for the mixed bacteria culture of at least one homofermentative lactic acid bacteria and at least one heterofermentative lactic acid bacteria, which culture, in correspondence with the diagram of FIG. 3, may be the inoculum 112.

A continuous feed fermenter 205 may be provided to receive the hydrolyzed biomass material and the mixed bacteria culture medium for fermentation of the hydrolyzed biomass material with the mixed bacteria in a resultant fermentation broth 114. The fermenter 205 may include a stirring device 207 for agitating the broth to improve contact between the bacteria and the biomass material.

A separation device 210 may be provided for receiving the fermentation broth from the fermenter and separating a lactic acid portion from the fermentation broth. Any simultaneously produced acetic acid may still be present with the lactic acid and may require further processing for separating the lactic acid from the acetic acid. In an embodiment, the separation device 210 may be a filter module that has at least one filter membrane with a pore size for retaining the hydrolyzed biomass material and the bacteria in the fermentation broth while allowing at least a portion of the liquid containing the lactic acid to pass through to a collection container 215.

The IPS system may have at least one pump for moving the liquid components though the system. The pump may feed hydrolyzed biomass material 104 from the supply reservoir 200 to the fermenter 205, may feed the mixed bacteria culture 112 to the fermenter, may feed fermentation broth 114 from the fermenter to the separator 210, may provide a return 220 of at least a portion of the fermentation broth from the separator back to the fermenter, or combinations thereof.

In an embodiment as depicted in FIG. 6, a separate pump 232 may feed hydrolyzed biomass material 104 from the supply reservoir 200 to the fermenter 205, or alternatively, if the reservoir is located above the fermenter, gravity flow may be used to feed hydrolyzed biomass material from the supply reservoir to the fermenter. Similarly, in an embodiment, a separate pump 234 may feed the mixed bacteria culture 112 to the fermenter 205, or alternatively, if the second reservoir 202 was located above the fermenter, gravity flow may be used to feed the mixed bacteria culture to the fermenter. In an embodiment, a pump 236 may be provided to feed fermentation broth 114 to the separator 210 and/or return 220 at least a portion of the fermentation broth from the separator back to the fermenter. In one embodiment, the pump 236 may have a flow rate of at least about 150 ml/min, and may have a flow rate of about 160 ml/min, or about 170 ml/min, or about 180 ml/min, or about 190 ml/min, or about 200 ml/min, or about 210 ml/min, or about 220 ml/min, or any flow rate between any of the listed values. In an embodiment, the overall flow rate of the system may be a function of the filtering capacity, the density of components in the broth, and other system parameters.

An IPS may also include a pH monitoring device 240 with a supply 250 of a pH adjusting solution which may be added as necessary to maintain the pH at a preferred operating value. For a lactic acid production, the pH may be about 5, and the pH adjusting solution may be sodium hydroxide. Alternatively, the pH may be about 4.0, about 4.2, about 4.4, about 4.6, about 4.8, about 5.2, about 5.4, about 5.6, about 5.8, or about 6.0, or any value between any of the listed values.

In an IPS, an essentially continuous fluid flow may be provided from the reservoir 200 to the container 215. The system may include flow rate monitors and fluid level monitors (not shown) and appropriate associated electronic controls and processing systems to adjust various flow rates and fluid levels to allow for essentially continuous output of product. After an initial inoculation of the bacteria inoculum 112 into the fermenter 205 and an input of some C5/C6 sugar mixture 104, a fermentation 116 (FIGS. 3-5) of the sugars may take place to convert the sugars into the desired final organic acid or biofuel product, which as illustrated in FIGS. 3-5 may be lactic acid. A draw off fermentation broth portion 118 may then contain undigested sugars, both live and dead bacteria cells, and organic acid product. The draw off portion 118 may be pumped to the separator module 210 for recovery of organic acid or biofuel product.

An IPS system may also be used in a semi-continuous fermentation, wherein, after an initial start-up, the system is allowed to ferment without any additional flow for a period of time to convert most of the available sugars. At the end of the fermentation time, the broth may then be pumped through the separation module for separation of the bacteria and any undigested sugars from the lactic acid product, with a return of the cells and sugars to the fermenter. After most of the broth has been filtered, a new batch of sugar may be added to the fermenter, which will then already contain bacteria and a new fermentation may be allowed to progress. The fermentation and filtration steps may be repeated.

In an embodiment as depicted in FIG. 3, the separator module 210 may have two filter members, an ultrafiltration module 120 and a nanofiltration module 122. The ultrafiltration module 120 may have a filter membrane with a pore size that is sufficient for retaining the bacteria cells in a first fermentation broth portion 124 while allowing the monosaccharides, and the lactic acid to pass through in a second fermentation broth portion 126. The cell portion 124 may be recycled back into the fermenter 205, while the sugar/lactic acid portion 126 may be conducted to the second nanofiltration module 122. The nanofiltration module 122 may have a filter membrane with a pore size that is sufficient for retaining the monosaccharides in a third fermentation broth portion 128 while allowing the lactic acid to pass through in a fourth fermentation broth portion 130 containing the organic acid product. The sugar portion 128 may be recycled back into the fermenter 205.

The membrane in the ultrafiltration module may have a molecular weight cutoff of about 1400 Da to about 1600 Da. In an embodiment, the molecular weight cutoff may be about 1400 Da, about 1420 Da, about 1440 Da, about 1460 Da, about 1480 Da, about 1500 Da, about 1520 Da, about 1540 Da, about 1560 Da, about 1580 Da, about 1600 Da, or any value between any of the listed values.

The membrane in the nanofiltration module may have a molecular weight cutoff of about 400 Da to about 600 Da. In an embodiment, the molecular weight cutoff may be about 400 Da, about 420 Da, about 440 Da, about 460 Da, about 480 Da, about 500 Da, about 520 Da, about 540 Da, about 560 Da, about 580 Da, about 600 Da, or any value between any of the listed values.

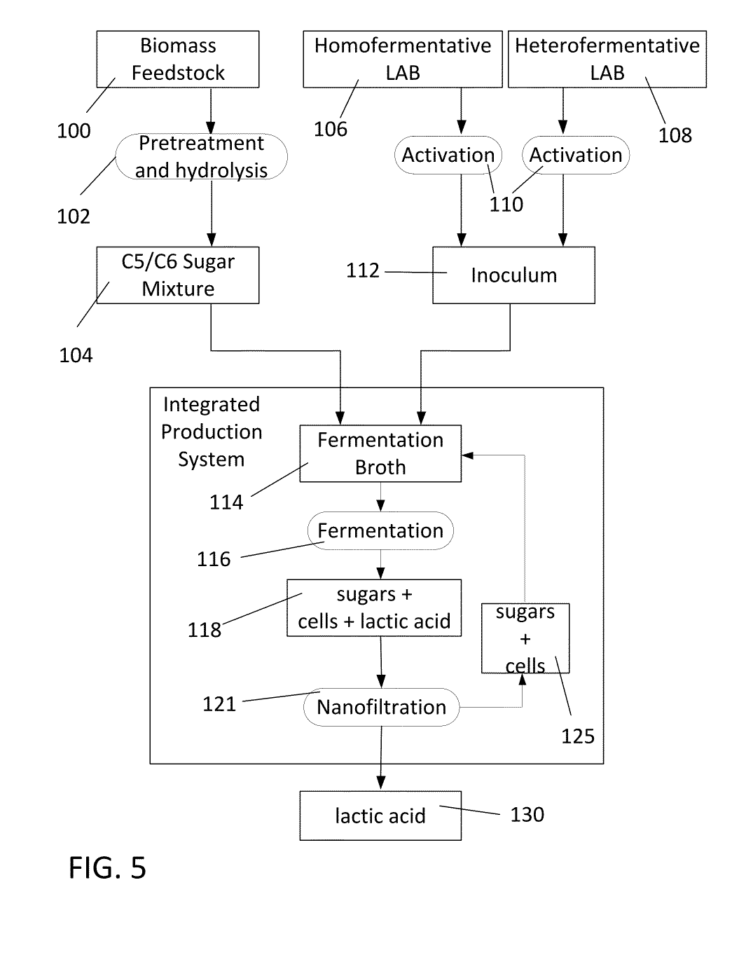

In an embodiment as depicted in FIG. 5, the separator 210 may have only a single nanofiltration membrane of the type discussed above, and having a pore size that is sufficient for retaining the bacteria cells and monosaccharides in a fermentation broth portion 125 while allowing the lactic acid to pass through in a fermentation broth portion 130. The cell/sugar portion 125 may be recycled back into the fermenter 205. In alternative embodiments, additional filter configurations may be used, which may include more than two filter membranes having decreasingly smaller pore sizes. Pore sizes may be configured on a basis of the ingredients in the fermentation broth and the desired content of filtered portions and passed though constituents.

After filtration in the separator module 210, the lactic acid portion 130 may contain additional unwanted constituents and contaminants, such as possible the co-produced by-product acetic acid. Thus, a further purification of the lactic acid may be performed. Lactic acid may be purified by adding calcium carbonate to the mixture to react with the lactic acid to form calcium lactate. In an embodiment, calcium carbonate may be used as the pH adjusting agent of supply 250. The calcium lactate may then be filtered from solution and dried. In an embodiment, lactic acid may be recovered by reacting the calcium lactate with sulfuric acid whereby calcium sulfate and lactic acid may be obtained. In an embodiment, further purification may be done by treating with carbon, filtration and evaporation.

For the production of lactic acid, the mixed bacteria culture may include at least one homofermentative lactic acid bacteria in any combination with at least one heterofermentative lactic acid bacteria. In an embodiment, the at least one homofermentative lactic acid bacteria may be one or more of Lactobacillus rhamnosus, Lactobacillus delbrueckii, Lactobacillus casei, Lactobacillus acideophilus, and Lactobacillus bulgaricus, and the at least one heterofermentative lactic acid bacteria may be one or more of Lactobacillus pentosus, Lactobacillus brevis, and Lactobacillus lactis. Some examples of combinations may include: Lactobacillus rhamnosus and Lactobacillus pentosus; or Lactobacillus rhamnosus and Lactobacillus brevis; or Lactobacillus delbrueckii, Lactobacillus casei and Lactobacillus lactis; or Lactobacillus rhamnosus, Lactobacillus delbrueckii, Lactobacillus brevis and Lactobacillus lactis; or essentially any combination of the listed bacteria species or other species which may breakdown cellulose and hemicellulose into their constituent monomers.

In an embodiment using an IPS with a mixed culture of Lactobacillus rhamnosus as the homofermentative lactic acid bacteria and Lactobacillus pentosus as the heterofermentative lactic acid bacteria, a production rate of lactic acid of about 2.5 g/L·h to about 3.1 g/L·h. may be achieved, which is an increase in productivity by a factor of about four over production with only a single species of either homofermentative bacteria or heterofermentative bacteria. Also, a yield of about 0.67 g to about 0.92 g lactic acid/g monosaccharides may be attained, which is an increase by a factor of about 1.1 over production with only a single species of either homofermentative bacteria or heterofermentative bacteria.

For batch production methods, in an embodiment with a mixed culture of Lactobacillus rhamnosus as the homofermentative lactic acid bacteria and Lactobacillus pentosus as the heterofermentative lactic acid bacteria, a production rate of lactic acid of about 0.5 g/L·h to about 0.89 g/L·h. may be achieved, which is an increase in productivity by a factor of about 20% over production with only a single species of either homofermentative bacteria or heterofermentative bacteria. And, a yield of about 0.73 g to about 0.89 g lactic acid/g monosaccharides may be attained, which is an increase by a factor of about 18% over production with only a single species of either homofermentative bacteria or heterofermentative bacteria.

With mixed culture fermentation, at least about 90% the hydrolyzed biomass material may be converted to lactic acid and acetic acid, and the amount of lactic acid produced may be about 10 times to about 20 times greater than the amount of acetic acid produced.

### Example 1

### A Kit of Mixed Bacteria Culture for Lactic Acid Production

A homofermentative lactic acid bacteria strain, Lactobacillus rhamnosus was cultivated to an actively growing and thriving culture in a Man Rogosa Sharpe media. Separately, a heterofermentative lactic acid bacteria strain, Lactobacillus pentosus, was cultivated to an actively growing and thriving culture in a Man Rogosa Sharpe media.

About 1 ml of sterile 30% glycerol was placed in 3 ml sealable test tubes. About 500 μl of each of the Lactobacillus rhamnosus and Lactobacillus pentosus cultures was placed in the tubes and the contents were mixed on a vortex mixer. The tubes were sealed and immediately frozen in liquid nitrogen to produce a frozen kit stock of a mixed bacteria culture for use in producing lactic acid.

### Example 2

### An Integrated Production System for Producing Lactic Acid

A first reservoir vessel 200 was configured for receiving a batch of C5/C6 sugars 104 and was outfitted with a pump 232 capable of pumping at variable rates of from 0 ml/min to about 300 ml/min. A second reservoir 202 was configured for receiving a mixed bacteria culture and was outfitted with a pump 234 having a variable flow rate. An outlet line was provided with a pump 236 capable of pumping at variable rates of from 0 ml/min to about 300 ml/min.

Pump 236 was connected to a separator module 210 which was equipped with a first ultrafiltration membrane with a molecular weight cutoff of about 1500 Da to filter out bacteria cells, and a second nanofiltration membrane with a molecular weight cutoff of about 500 Da to filter out undigested sugars and allow filtrate containing the final product to pass through into a container 215. The total effective area of the filters was about 140 cm². A return line 220 was also connected from the filter module 210 to the fermenter 205 for returning undigested sugars and bacteria back to the fermenter.

### Example 3

### Integrated Production Fermentation

An integrated production system using the mixed culture of Example 1 and having the components of Example 2 was used for producing lactic acid from non-food biomass feedstock. A biomass feedstock of wheat straw was pretreated to break down any lignins which were present in the biomass. The pretreatment was done by mixing the biomass with 1.0 M sulfuric acid at a ratio of about 1000 liters H₂SO₄ per 100 kg biomass. The mixture was heated to about 130° C. for about 1 hour. The biomass material was filtered from the solution, rinsed to remove any remaining acid, and placed in a reaction vessel for hydrolysis.

For hydrolysis, the biomass material was mixed with water at a ratio of about 1000 liters H₂O per 30˜50 kg biomass. The specialized enzyme mixture Spezyme® CP (cellulase activity 50 PU/ml) was added at a ratio of about 50 ml Spezyme per 100 kg biomass, and the mixture was reacted, with stirring, at 35˜50° C. or about 12 hours. The resultant C5/C6 sugar mixture 104 was placed in a first reservoir vessel 200.

A mixed bacteria inoculum 112 was prepared from a kit stock of Example 1 of Lactobacillus rhamnosus and Lactobacillus pentosus. A 3 ml kit test tube of mixed bacteria was obtained and thawed, and the culture was activated by placing the culture in 3000 ml of Man Rogosa Sharpe media. The culture was propagated for about 16 hours to prepare the inoculum 112 and was placed into the second reservoir vessel 202.

Fermentation in the IPS was then initiated by pumping about 60% of the working fermenter volume of the C5/C6 sugar mix 104 into the fermenter 205. The pH was adjusted to about 5.0 with calcium carbonate and the fermenter was inoculated with about 5% of culture media volume of mixed bacteria inoculum 112. After an initial start-up time of about 24 hours to allow a substantial portion of the original sugars to be converted to lactic acid, the continuous production process was started by initiating operation of the pumps 232, 234, 236 to move fluids through the system. About 200 ml/min of the fermentation broth 114 was pumped through the separator module 210 to continuously collect lactic acid in container 215.

Fermentation broth containing undigested sugars and bacterial cells was recycled back into the fermenter 205. Pump 232 was controlled and operated as needed to maintain a substantially constant level of fermentation broth 114 in the fermenter 205, and pump 234 was operated based on the usable life-span of the bacteria to maintain an optimum working bacteria culture in the fermenter.

With this system, a lactic acid yield of from about 0.67 g to about 0.92 g lactic acid per gram of sugar was achieved which is about 1.1 times that achievable with other systems. In addition, a volumetric productivity of about 2.5 g to about 3.1 g lactic acid per Liter-hour of broth processed was achieved, which is about 4 times greater than that achievable with other systems.

### Example 4

### Comparison of Mixed Culture Vs. Single Culture Fermentation

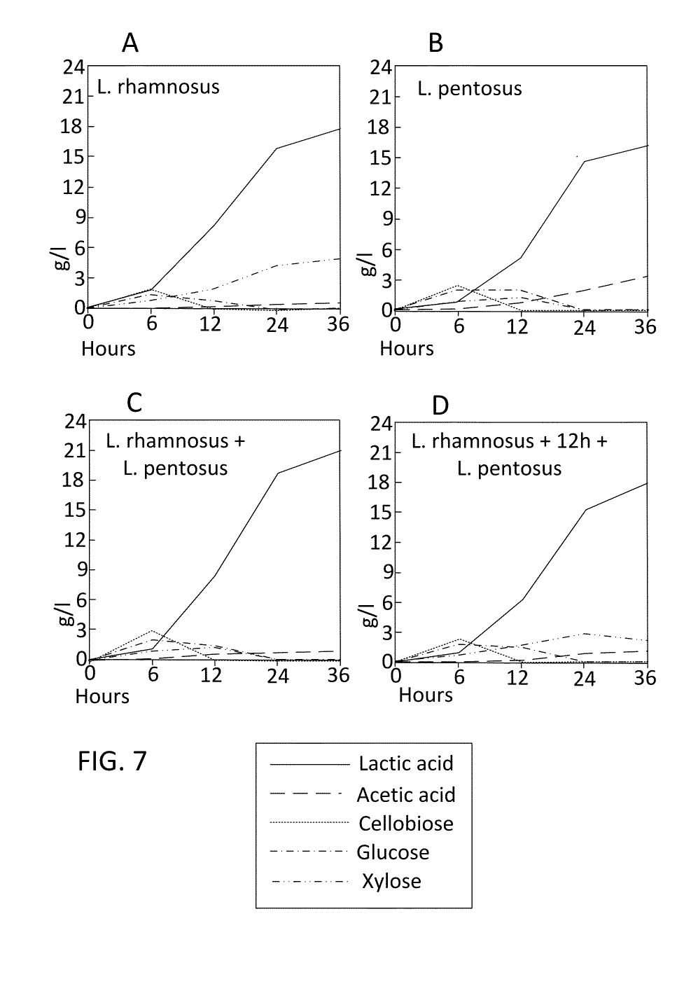

A comparison of yields of lactic acid and acetic acid as well as consumption of glucose and xylose was conducted with a batch fermentation with corn stover as the biomass material. A fermentation broth was prepared in a 100 liter fermenter by adding about 1.8 kg of hydrolyzed corn stover to about 60 liters of water. The pH was adjusted to about 5.0. This was followed with about 5 mL of Spezyme and 300 ml of bacteria culture for converting the sugars. The bacteria cultures included: FIG. 7A—only Lactobacillus rhamnosus; FIG. 7B—only Lactobacillus pentosus; FIG. 7C—simultaneous mixed culture of Lactobacillus rhamnosus and Lactobacillus pentosus; and FIG. 7D—only Lactobacillus rhamnosus for 12 hours followed by Lactobacillus pentosus.

Samples were taken at 6, 12, 24 and 36 hours and were analyzed for cellobiose, glucose, xylose, lactic acid and acetic acid. The mixed culture of Lactobacillus rhamnosus and Lactobacillus pentosus produced the highest yield of lactic acid of about 20.95 g/l and only about 1.87 g/l acetic acid with almost complete utilization of the sugars. The single culture of Lactobacillus pentosus utilized the sugars well but produced less lactic acid, 16.71 g/l and excess acetic acid, 3.1 g/l, while the single culture of Lactobacillus rhamnosus produced more lactic acid 17.70 g/l than did the Lactobacillus pentosus the lactic acid was less than the mixed culture as the utilization of the xylose was poor.

### Example 5

### Semi-Continuous Fermentation

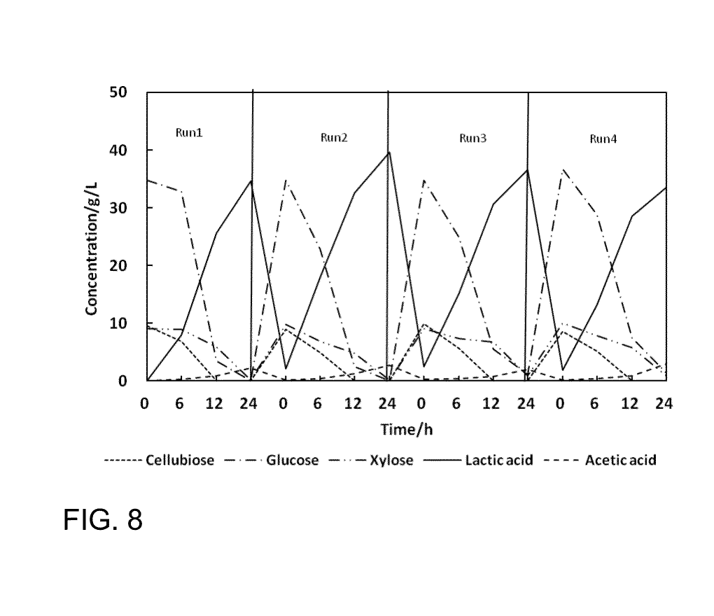

A 4-run semi-continuous fermentation for lactic acid production using the Integrated Production System (IPS) was conducted with corn stover as the biomass material. About 3.0 kg of pretreated corn stover in about 60 liters of water was hydrolyzed with about 8 mL of Spezyme for about 12 hours. The pH was adjusted to about 5.0. Fermentation in the IPS was then initiated by pumping about 60% of the working fermenter volume of the C5/C6 sugar mix 104 into the fermenter 205. The pH was adjusted to about 5.0 with calcium carbonate and the fermenter was inoculated with about 5% of culture media volume of mixed bacteria inoculum 112 to start fermentation. Samples of the broth were taken at 0, 6, 12 and 24 hours and were analyzed for cellobiose, glucose, xylose, lactic acid and acetic acid (see results below and in FIG. 8). After a fermentation period of about 24 hours to allow a substantial portion of the original sugars to be converted to lactic acid, the semi-continuous production process was started by initiating operation of the pump 236 to move fluids through the system. About 200 ml/min of the fermentation broth 114 was pumped through the separator module 210 to continuously collect lactic acid in container 215. Filtering was done for at least about 4 hours for processing/filtering about 54 liters of broth.

A second fermentation was started by adding about 60 liters of freshly obtained C5/C6 sugar mix 104 from the first reservoir vessel 200 into the fermenter 205 by pump 232. After about 24 hours, the broth was again filtered for lactic acid collection in the manner as discussed above. Two additional fermentation and filtering cycles were done.

Samples were taken at 0, 6, 12 and 24 hours and were analyzed for cellobiose, glucose, xylose, lactic acid and acetic acid. About 36 g/L of lactic acid and 2.3 g/L of acetic acid were produced in each experimental run by using the mixed culture of Lactobacillus rhamnosus and Lactobacillus pentosus with complete consumption of C5/C6 sugar mix. A total of about 8666.8 g of lactic acid and 601.4 g of acetic acid were produced in the 100-L scale fermenter during this 4-run semi-continuous fermentation.

| | | | | | | | Total | Total |
|---|---|---|---|---|---|---|---|---|
| | Time | Cellobiose | Glucose | Xylose | Lactic acid | Acetic acid | Lactic acid | Acetic acid |
| Run | (hours) | (g/l) | (g/l) | (g/l) | (g/l) | (g/l) | (g) | (g) |
| 1 | 0 | 9.54 | 34.78 | 9.02 | 0.00 | 0.00 | | |
| | 6 | 6.91 | 32.87 | 8.91 | 7.95 | 0.27 | | |
| | 12 | 0.00 | 3.50 | 5.87 | 25.60 | 0.92 | | |
| | 24 | 0.00 | 0.01 | 0.21 | 34.62 | 2.28 | 2077.00 | 137.00 |
| 2 | 0 | 9.00 | 34.78 | 9.82 | 2.10 | 0.20 | | |
| | 6 | 4.91 | 22.87 | 6.91 | 17.95 | 0.37 | | |
| | 12 | 0.00 | 2.50 | 4.87 | 32.60 | 1.22 | | |
| | 24 | 0.00 | 0.01 | 0.21 | 39.61 | 2.78 | 2376.60 | 166.80 |
| 3 | 0 | 9.94 | 34.78 | 9.02 | 2.50 | 0.31 | | |
| | 6 | 5.71 | 24.87 | 7.31 | 15.20 | 0.47 | | |
| | 12 | 0.00 | 5.50 | 6.77 | 30.60 | 0.82 | | |
| | 24 | 0.00 | 1.20 | 0.90 | 36.61 | 1.98 | 2196.60 | 118.80 |
| 4 | 0 | 8.54 | 36.78 | 10.02 | 1.90 | 0.21 | | |
| | 6 | 5.21 | 28.87 | 7.91 | 13.20 | 0.47 | | |
| | 12 | 0.00 | 7.50 | 5.77 | 28.60 | 0.95 | | |
| | 24 | 0.00 | 1.40 | 0.90 | 33.61 | 2.98 | 2016.60 | 178.80 |
| | | | | | | | 8666.80 | 601.40 |

This disclosure is not limited to the particular systems, devices and methods described, as these may vary. The terminology used in the description is for the purpose of describing the particular versions or embodiments only, and is not intended to limit the scope.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method for producing lactic acid from biomass material, the method comprising:
hydrolyzing the biomass material to form a mixture of monosaccharides;
fermenting the monosaccharides in a fermenter with a fermentation broth of a mixed bacteria culture to produce lactic acid and acetic acid, the mixed bacteria culture comprising:
at least one homofermentative lactic acid bacteria comprising Lactobacillus rhamnosus, Lactobacillus delbrueckii, Lactobacillus casei, Lactobacillus acideophilus, Lactobacillus bulgaricus, or combinations thereof; and
at least one heterofermentative lactic acid bacteria comprising Lactobacillus pentosus Lactobacillus brevis, Lactobacillus lactis or combinations thereof;
converting at least about 90% of the monosaccharides to lactic acid and acetic acid;
recovering lactic acid from the fermentation broth at a yield of at least about 0.65 gram lactic acid per gram of monosaccharides; and
recovering acetic acid at a yield of at most about 0.065 gram acetic acid per gram of monosaccharides.

2. (canceled)

3. The method of claim 1, wherein the at least one homofermentative lactic acid bacteria comprises Lactobacillus rhamnosus, and the at least one heterofermentative lactic acid bacteria comprises Lactobacillus pentosus.

4. The method of claim 1, further comprising producing lactic acid at a productivity rate of at least about 0.50 gram per Liter-hour to about 0.89 gram per Liter-hour.

5. (canceled)

6. The method of claim 1, wherein the fermenting comprises:
fermenting the monosaccharides in a first fermentation broth with only the at least one homofermentative bacteria to convert a first portion of the monosaccharides into lactic acid;
adding the at least one heterofermentative bacteria to the first fermentation broth to form a second fermentation broth; and
fermenting additional monosaccharides in the second fermentation broth with both the at least one homofermentative bacteria and the at least one heterofermentative bacteria to convert an additional portion of the monosaccharides into lactic acid.

7. The method of claim 1, wherein:
hydrolyzing the biomass material comprises:
contacting the biomass material and at least one cellulase to convert at least a cellulose portion of the biomass material into glucose;
wherein the cellulase comprises endocellulase (EC 3.2.1.4), exocellulase (EC 3.2.1.91) and cellobiase (EC 3.2.1.21);
contacting the biomass material and at least one xylanase to convert at least a hemicellulose portion of the biomass material into xylose, glucose and at least one of glucuronic acid, mannose, galactose, rhamnose, and arabinose; and
wherein the xylanase comprises endo-1,4-β-xylanase (EC 3.2.1.8), exo-1,3-β-xylosidase (EC 3.2.1.72), and exo-1,4-β-xylosidase (EC 3.2.1.37).

8-10. (canceled)

11. The method of claim 1, wherein the method further comprises pre-treating the biomass material prior to the hydrolyzing to break down and remove lignins from the biomass material.

12. (canceled)

13. The method of claim 11, wherein the pre-treating comprises contacting the biomass material with about 0.5 M to about 1.5 M of at least one of sulfuric acid, hydrochloric acid and nitric acid at a temperature from about 80° C. to about 150° C. for about 2 to about 80 minutes.

14. (canceled)

15. The method of claim 11, wherein the pre-treating comprises contacting the biomass material with about 0.1M to about 2.0 M of at least one of sodium hydroxide, potassium hydroxide or calcium hydroxide at temperature from about 25° C. to about 60° C. for about 0.5 to about 5 hours.

16. (canceled)

17. The method of claim 1, further comprising:
introducing a flow of the monosaccharides into the fermentation broth in the fermenter;
fermenting the monosaccharides in the fermenter; and
continuously recovering lactic acid from the fermenter at a production rate of at least about 2.5 gram per Liter-hour.

18. (canceled)

19. The method of claim 1, wherein recovering the lactic acid comprises:
passing the fermentation broth through a filter having a pore size for retaining undigested monosaccharides, at least one homofermentative lactic acid bacteria and the at least one heterofermentative lactic acid bacteria in the fermentation broth while allowing a liquid broth portion comprising the lactic acid to pass through;
collecting the liquid broth portion; and
purifying the lactic acid from the liquid broth portion by:
adding calcium carbonate to the liquid broth portion to form calcium lactate;
filtering calcium lactate from the liquid broth portion;
reacting the calcium lactate with sulfuric acid to form lactic acid and calcium sulfate; and
filtering the calcium sulfate out of the lactic acid.

20-21. (canceled)

22. The method of claim 1, further comprising:
introducing, at least periodically, a flow of the monosaccharides into the fermentation broth in the fermenter;
pumping the fermentation broth through a filter module comprising a membrane having a pore size for retaining the monosaccharides, the at least one homofermentative lactic acid bacteria and the at least one heterofermentative lactic acid bacteria in a first fermentation broth portion while allowing the lactic acid to pass through in a second fermentation broth portion;
separating the second fermentation broth portion from the first fermentation broth portion in the filter module;
returning the first fermentation broth portion to the fermenter;
collecting the second fermentation broth portion in a collection vessel; and
purifying the lactic acid from the second fermentation broth portion.

23. The method of claim 1, further comprising:
introducing, at least periodically, a flow of the monosaccharides into the fermentation broth in the fermenter;
pumping the fermentation broth through a filtration system comprising first and second filter modules;
separating a second fermentation broth portion from a first fermentation broth portion in the first filter module comprising a membrane having a pore size for retaining the at least one homofermentative lactic acid bacteria and the at least one heterofermentative lactic bacteria in a first fermentation broth portion while allowing the monosaccharides, and the lactic acid to pass through in the second fermentation broth portion;
separating a fourth fermentation broth portion from a third fermentation broth portion in the second filter module comprising a membrane having a pore size for retaining the monosaccharides in the third fermentation broth portion while allowing the lactic acid to pass through in the fourth fermentation broth portion;
returning the first and third fermentation broth portions to the fermenter;
collecting the fourth fermentation broth portion in a collection vessel; and
purifying the lactic acid from the second fermentation broth portion.

24. The method of claim 23, wherein:
the at least one homofermentative lactic acid bacteria comprises Lactobacillus rhamnosus, and the at least one heterofermentative lactic acid bacteria comprises Lactobacillus Pentosus;
the fermenter comprises a continuous feed fermenter with continuous recovery of lactic acid; and
the method further comprises:
introducing a flow of the monosaccharides into the fermentation broth in the fermenter;
fermenting the monosaccharides in the fermenter; and
continuously recovering lactic acid from the fermenter at a production rate of at least about 2.5 gram per Liter-hour.

25. The method of claim 1, wherein:
the method further comprises pre-treating the biomass material prior to the hydrolyzing to break down and remove lignins from the biomass material; and
the hydrolyzing comprises contacting the pre-treated biomass material and at least one cellulase to break down the biomass material into the monosaccharides.

26-28. (canceled)

29. The method of claim 1, wherein the biomass comprises at least one of sawdust, corn stover, wheat straw, rice straw, switchgrass, bagasse, poplar wood, paper mill waste and municipal paper waste.

30-31. (canceled)

32. A method for producing lactic acid from non-food biomass material, the method comprising:
hydrolyzing non-food biomass material to form a mixture of monosaccharides;
fermenting the monosaccharides in a fermenter with a fermentation broth of a mixed bacteria culture to produce lactic acid and co-produced acetic acid, the mixed bacteria culture consisting essentially of Lactobacillus rhamnosus, a homofermentative lactic acid bacteria, and Lactobacillus pentosus, a heterofermentative bacteria; and
recovering lactic acid from the fermentation broth.

33. (canceled)

34. The method of claim 32, further comprising:
converting at least about 90% of the monosaccharides into lactic acid; and
producing lactic acid at a yield of at least about 10 times greater than a yield of acetic acid produced, and at least about 16% greater than a yield of lactic acid produced by fermentation with only a single species of either homofermentative bacteria or heterofermentative bacteria.

35. (canceled)

36. A system for producing lactic acid from biomass material, the system comprising:
a first supply reservoir configured for providing hydrolyzed biomass material;
a second supply reservoir configured for providing a mixed bacteria culture medium of at least one homofermentative lactic acid bacteria and at least one heterofermentative lactic acid bacteria;
a fermenter configured for receiving the hydrolyzed biomass material and the mixed bacteria culture medium for fermentation of the hydrolyzed biomass material with the mixed bacteria in a resultant fermentation broth;
a filter configured for receiving the fermentation broth from the fermenter and separating from the fermentation broth any lactic acid and acetic acid produced by the at least one homofermentative lactic acid bacteria and the at least one heterofermentative lactic acid bacteria;
a collector configured for receiving the lactic acid and acetic acid from the filter; and
at least one pump for feeding hydrolyzed biomass material from the supply reservoir to the fermenter, feeding the mixed bacteria culture to the fermenter, feeding fermentation broth from the fermenter to the filter, returning at least a portion of the fermentation broth from the filter back to the fermenter, or combinations thereof.

37-41. (canceled)

42. The system of claim 36, wherein:
the mixed bacteria culture medium comprises both Lactobacillus rhamnosus and Lactobacillus pentosus;
the system comprises a continuous flow, non-batch system configured for continuous output of lactic acid from fermenting biomass material; and
the productivity of lactic acid is at least about 2.5 gram per Liter-hour, wherein substantially all of the hydrolyzed biomass material is converted to lactic acid and acetic acid, and the amount of lactic acid produced is at least about 10 times greater than the amount of acetic acid produced.

43-46. (canceled)

47. The system of claim 36, wherein:
the fermentation broth comprises the hydrolyzed biomass material, bacteria, and a liquid component comprising the lactic acid and acetic acid; and
the filter comprises a flow through filter module comprising at least one filter membrane having a pore size for retaining the hydrolyzed biomass material and the bacteria in the portion of the fermentation broth returned to the fermenter while allowing at least a portion of the liquid component to pass through to the collector.

48. The system of claim 36, wherein:
the fermentation broth comprises the hydrolyzed biomass material, bacteria, and a liquid component comprising the lactic acid and acetic acid; and
the filter module comprises a flow through filter module comprising:
a first membrane for receiving fermentation broth from the fermenter and having a pore size for retaining the bacteria in a first fermentation broth portion while allowing the hydrolyzed biomass material and a first portion of the liquid component to pass through;
wherein the first membrane has a molecular weight cutoff of about 1500 Da;
a second membrane for receiving the first portion of the liquid component and having a pore size for retaining the hydrolyzed biomass material in a second fermentation broth portion while allowing at least an additional portion of the liquid component to pass through to the collector;
wherein the second membrane has a molecular weight cutoff of about 500 Da; and
a return for returning the first fermentation broth portion and the second fermentation broth portion to the fermenter.

49. (canceled)

50. The system of claim 36, wherein the at least one pump comprises:
at least a first pump for feeding hydrolyzed biomass material from the first supply reservoir to the fermenter;
at least one second pump for feeding the mixed bacteria culture medium from the second supply reservoir to the fermenter; and
at least one third pump for feeding the fermentation broth from the fermenter to the filter and returning the at least a portion of the fermentation broth from the filter back to the fermenter.

51-58. (canceled)

59. The system of claim 36, wherein:
the biomass material comprises non-food biomass material; and
the system further comprises:
a pre-treatment vessel for pre-treating non-food biomass material to break down and remove lignins from the non-food biomass material, the pre-treatment vessel comprising at least one of:
an acid treatment vessel for treatment with about 0.5 M to about 1.5 M of at least one of sulfuric acid, hydrochloric acid and nitric acid at a temperature from about 80° C. to about 150° C. for about 2 to about 80 minutes; and
a base treatment vessel for treatment with about 0.1 M to about 2.0 M of at least one of sodium hydroxide, potassium hydroxide or calcium hydroxide at temperature from about 25° C. to about 60° C. for about 0.5 to about 5 hour; and
a hydrolysis vessel for receiving pre-treated, non-food biomass material and at least one cellulase for hydrolysis of the pre-treated, no-food biomass material into glucose, xylose and at least one additional pentose.

60. The system of claim 36, wherein
the system is a continuous flow, non-batch system configured for continuous output of lactic acid from fermenting biomass material.

61. The system of claim 60, wherein
the filter comprises:
a first flow through module for receiving fermentation broth from a fermenter, the first module comprising a first membrane having a molecular weight cutoff of about 1500 Da for retaining the bacteria in a first fermentation broth portion while allowing the hydrolyzed biomass material and a first portion of the liquid component to pass through;
a second flow through module for receiving the hydrolyzed biomass material and the first portion of the liquid component from the first module, the second module comprising a second membrane having a molecular weight cutoff of about 500 Da for retaining the hydrolyzed biomass material in a second fermentation broth portion while allowing a portion of the liquid component to pass through to the collector; and
a return for returning the first fermentation broth portion and the second fermentation broth portion to the fermenter.

62-67. (canceled)
